(19) United States
(12) Patent Application Publication
(10) Pub. No.: US 2025/0186209 A1
(43) Pub. Date: Jun. 12, 2025

(54) ECHOGENIC SUTURES FOR CARDIAC PROCEDURES

(71) Applicant: EDWARDS LIFESCIENCES CORPORATION, Irvine, CA (US)

(72) Inventors: Felino V. Cortez, JR., Bowie, MD (US); Stephen Cournane, Severn, MD (US); Sandip Vasant Pawar, Irvine, CA (US); Gillian Irene Armstrong, Huntington Beach, CA (US); Luke Anthony Zanetti, Parkton, MD (US); Stephen Epstein, Millersville, MD (US); Ashley Nicolette Hinga, Eldersburg, MD (US); Nancy Ling Chung, Edgewater, MD (US)

(21) Appl. No.: 19/057,596

(22) Filed: Feb. 19, 2025

Related U.S. Application Data
(63) Continuation of application No. PCT/US2023/029575, filed on Aug. 4, 2023.
(60) Provisional application No. 63/371,912, filed on Aug. 19, 2022.

(51) Int. Cl. A61F 2/24; A61L 27/16; A61L 27/50

(52) U.S. Cl. CPC A61F 2/2457; A61L 27/16; A61L 27/50; A61F 2210/0009; A61F 2210/0057; A61F 2210/0076; A61F 2250/0015; A61L 2430/20

(57) ABSTRACT

Disclosed herein are sutures (or cords) and methods for using sutures wherein the sutures comprise a material or a combination of materials configured to increase echogenicity of the sutures. In some examples, the sutures comprise echogenic materials incorporated into suture material (e.g., ePTFE). In some examples, the sutures comprise a combination of materials with abrupt and relatively large changes in density to increase reflections of ultrasound waves. The disclosed sutures enhance echogenicity to increase visibility of the sutures using ultrasound imaging technologies. Furthermore, disclosed herein are methods that utilize the disclosed sutures in valve repairs including implanting artificial cords, sub-valvular techniques, reshaping organs, annuloplasty, and the like. Utilization of the disclosed echogenic sutures can result in superior outcomes due at least in part to physicians being able to better visualize the placement and/or tension of the echogenic sutures.

### CROSS-REFERENCE TO RELATED APPLICATIONS

This application is a continuation of International Patent Application No. PCT/US2023/029575, filed on Aug. 4, 2023, which claims the benefit of U.S. Patent Application No. 63/371,912 filed on Aug. 19, 2022, the entire disclosures all of which are incorporated by reference for all purposes.

### BACKGROUND

### Field

Some examples described herein relate to the use of echogenic sutures for cardiac procedures.

### Description of Related Art

Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the geometry of the heart causing valves in the heart to dysfunction. The vast majority of patients undergoing valve surgery, such as mitral valve surgery, suffer from a degenerative disease that causes a malfunction in a leaflet of the valve, which results in prolapse and regurgitation.

Valve regurgitation occurs when the leaflets of the valve do not close completely thereby allowing blood to leak back into the prior chamber when the heart contracts. This may be caused by dilation of the annulus (Carpentier type I malfunction), prolapse of a segment of one or both leaflets above the plane of coaptation (Carpentier type II malfunction), or restriction of the motion of one or more leaflets such that the leaflets are abnormally constrained below the level of the plane of the annulus (Carpentier type II malfunction). Mitral valve regurgitation (MR) results in a volume overload on the left ventricle which in turn progresses to ventricular dilation, decreased ejection performance, pulmonary hypertension, symptomatic congestive heart failure, atrial fibrillation, right ventricular dysfunction and death.

Malfunctioning valves may either be repaired or replaced. Replacement typically involves replacing the patient's malfunctioning valve with a biological or mechanical substitute. Repair typically involves the preservation and correction of the patient's own valve. Successful surgical mitral valve repair restores mitral valve competence, abolishes the volume overload on the left ventricle, improves symptom status, and prevents adverse left ventricular remodeling.

In many instances of mitral valve regurgitation, repair is preferable to valve replacement. Many surgeons have moved to a “non-resectional” repair technique where artificial chordae tendineae (“cords”) made of expanded polytetrafluoroethylene (“ePTFE”) suture, or another suitable material, are placed in the prolapsed leaflet and secured to the heart in the left ventricle, normally to the papillary muscle. Another technique, developed by Dr. Alfieri, involves securing the midpoint of both leaflets together to create a double orifice valve, known as an “edge-to-edge” repair or an Alfieri procedure. Another technique, in addition to or instead of creating the edge-to-edge relationship, includes securing together sutures extending from the leaflets to pull or to otherwise move the posterior annulus towards the anterior leaflet and/or the anterior annulus towards to posterior leaflet to reduce the distance between the anterior annulus and the posterior annulus (or the septal-lateral distance).

Although mitral valve repairs may utilize sutures, other cardiac procedures also utilize sutures to repair other cardiac valves, reshape portions of the heart, and/or to affect functionality of a targeted portion of the heart. Sutures may also be used in other procedures that aim to address issues in other organs or to help fix tissue such as tendons, ligaments, and the like.

### SUMMARY

Described herein are echogenic sutures or cords for surgical procedures such as cardiac procedures. As an example, the sutures comprising the materials and combinations of materials described herein can be implanted as artificial cords (e.g., replacing or supplementing native chordae tendineae) for repairing cardiac valves. The disclosed sutures are configured to amplify the echogenic signature of the sutures to increase visibility using ultrasound technology. The echogenic sutures comprise materials or combinations of materials that enhance echogenicity such as by including echogenic materials (e.g., micro bubbles, nanoparticles, glass beads, glass microspheres, etc.) and/or by including combinations of materials that form acute changes in density. In some instances, echogenic materials may be included in a polymer, in strands of materials wound with other suture material to form a suture, or the like. In certain implementations, the sutures include cord material that is braided or twisted and/or may include core material within the braided or twisted material. In various implementations, the sutures comprise a plurality of ePTFE strands braided together that may include one or more strands that has been modified to include echogenic material. In some instances, the cord material includes a core made of a synthetic aramid, a high-strength polymer such as polyethylene terephthalate (PET), a ceramic, and/or a metal with a coating of ePTFE or other suitable material.

In some implementations, the disclosed sutures include echogenic material embedded throughout a polymer. In some implementations, the disclosed sutures include echogenic material embedded on a surface of a polymer. In various implementations, the disclosed sutures include a core of a polymer that is surrounded by an echogenic material and, in certain instances, the echogenic material may be surrounded by an outer polymer layer. In some implementations, the disclosed sutures include a core of echogenic material surrounded by a polymer. In some implementations, the disclosed sutures include an echogenic material that is braided together with other material. In some implementations, the disclosed sutures include an echogenic material embedded between braids forming the suture. In some implementations, the disclosed sutures include a first material of a first density as a core and a second material of a second density as an outer layer, the first density being less than the second density. In these implementations, the disclosed sutures are configured to enhance echogenicity and/or to increase the reflections of ultrasound waves off of the sutures to enhance visualization using ultrasound imaging techniques.

In an aspect, the present disclosure provides an echogenic suture or echogenic cord used for medical procedures wherein an echogenic signal is augmented by acute changes in material density of the echogenic cord. In some implementations, the echogenic cord includes two or more materials wherein a change in density between adjacent materials is relatively large or acute to increase echogenic signals. In some implementations, the echogenic cord includes echogenic materials embedded in the echogenic cord.

In some implementations, an echogenic cord can be attached to targeted tissue of a heart. In some implementations, the echogenic cord includes a distal anchor and a suture extending proximally from the distal anchor implant. In some implementations, the echogenic cord has an echogenic material that increases visibility to an ultrasound imaging device. In some implementations, the proximal end of the echogenic cord can be anchored to the heart.

In some implementations, the echogenic cord comprises a polymer with echogenic material embedded therein. In some implementations, the echogenic cord comprises a polymer with echogenic material embedded on an outer surface of the polymer. In some implementations, the echogenic cord comprises an inner polymer core surrounded by an echogenic material layer that is surrounded by an outer polymer layer. In some implementations, the echogenic cord comprises an inner polymer core surrounded by an echogenic material layer. In some implementations, the echogenic cord comprises an echogenic material core surrounded by a polymer layer. In some implementations, the echogenic cord comprises a strand with echogenic material braided together with strands of polymers to form a braided echogenic cord. In some implementations, the echogenic cord comprises a plurality of strands of a polymer weaved together to form a braid, wherein echogenic material is trapped in the weaves of the braid. In some implementations, the echogenic cord comprises an inner core with a first density and an outer jacket with a second density, wherein a change in density between the first density and the second density provides an acute density change to increase ultrasound reflections off of the echogenic cord. In some implementations, the echogenic cord comprises one or more strands of a polymer and one or more strands of an echogenic material, the one or more strands of the polymer and the one or more strands of the echogenic material being plied together to form the echogenic chord. In some implementations, the echogenic cord comprises an ePTFE core surrounded by a jacket that includes echogenic material, the jacket formed from ribbons of a polymer with the echogenic material that are wrapped around the ePTFE core.

In some implementations, anchoring the proximal end includes securing the proximal end to an external wall of the heart. In some implementations, anchoring the proximal end includes securing the proximal end to a papillary muscle of the heart. In some implementations, the targeted tissue includes a leaflet of a mitral valve. In some implementations, the distal anchor is a bulky knot formed using the echogenic cord. In some implementations, the distal anchor is a barb secured to a distal end of the echogenic cord.

In some implementations, an echogenic suture for use in medical procedures is presented, the echogenic suture including materials with acute changes in density to augment an echogenic signal.

In some implementations, the echogenic suture comprises a polymer with echogenic material embedded therein. In some implementations, the echogenic suture comprises a polymer with echogenic material embedded on an outer surface of the polymer. In some implementations, the echogenic suture comprises an inner polymer core surrounded by an echogenic material layer that is surrounded by an outer polymer layer. In some implementations, the echogenic suture comprises an inner polymer core surrounded by an echogenic material layer. In some implementations, the echogenic suture comprises an echogenic material core surrounded by a polymer layer. In some implementations, the echogenic suture comprises a strand with echogenic material braided together with strands of polymers to form a braided echogenic suture. In some implementations, the echogenic suture comprises a plurality of strands of a polymer weaved together to form a braid, wherein echogenic material is trapped in the weaves of the braid. In some implementations, the echogenic suture comprises an inner core with a first density and an outer jacket with a second density, wherein a change in density between the first density and the second density provides an acute density change to increase ultrasound reflections off of the echogenic suture. In some implementations, the echogenic suture comprises one or more strands of a polymer and one or more strands of an echogenic material, the one or more strands of the polymer and the one or more strands of the echogenic material being plied together to form the echogenic chord. In some implementations, the echogenic suture comprises an ePTFE core surrounded by a jacket that includes echogenic material, the jacket formed from ribbons of a polymer with the echogenic material that are wrapped around the ePTFE core.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

FIG. 1 illustrates a cut-away anterior view of a heart, showing the internal chambers, valves and adjacent structures.

FIG. 2A illustrates a top perspective view of a healthy mitral valve with the mitral leaflets closed.

FIG. 2B illustrates a top perspective view of a dysfunctional mitral valve with a visible gap between the mitral leaflets.

FIG. 2C illustrates a cross-sectional view of a heart illustrating a mitral valve prolapsed into the left atrium.

FIG. 2D illustrates an enlarged view of the prolapsed mitral valve of FIG. 2C.

FIG. 3 illustrates a cross-sectional view of a heart showing the left atrium, right atrium, left ventricle, right ventricle and the apex region.

FIG. 4 illustrates a valve repair using an echogenic suture or cord.

FIG. 5 is a schematic illustration of using echogenic cords with bulky knots as anchors to repair a mitral valve with leaflets that are separated by a gap.

FIG. 6 illustrates an example of an annuloplasty ring that has a core surrounded by a jacket, the core comprising any of the echogenic sutures disclosed herein.

FIG. 7 illustrates an example sub-valvular procedure using any of the echogenic sutures disclosed herein.

FIG. 8 illustrates using an echogenic suture in a procedure to reshape a portion of the heart.

FIG. 9 illustrates using an echogenic suture in an annuloplasty procedure that implants the cord in the coronary sinus to reshape the annulus.

FIG. 10 illustrates using an echogenic suture in an annuloplasty procedure that implants a plurality of anchors in an annulus and pulls the anchors together using the echogenic suture to reshape the annulus.

FIG. 11 illustrates using an echogenic suture for the fixation of an implantable cardiac device.

FIG. 12 illustrates using an echogenic suture as an ultrasound detectable marker of a graft site.

FIG. 13 illustrates an example echogenic suture that includes echogenic material embedded throughout a polymer.

FIG. 14 illustrates an example echogenic suture that includes echogenic material embedded on a surface of a polymer.

FIG. 15 illustrates an example echogenic suture that includes an inner layer that is coated with echogenic material embedded surrounded by a polymer.

FIG. 16 illustrates an example echogenic suture that includes a polymer fully coated by echogenic material.

FIG. 17 illustrates an example echogenic suture that includes an echogenic material surrounded by a polymer layer.

FIG. 18 illustrates an example echogenic suture that includes a strand of echogenic material weaved between braids of the suture.

FIG. 19 illustrates an example echogenic suture that includes an echogenic material embedded between braids of the suture.

FIG. 20 illustrates an example echogenic suture that includes an acute density change between a higher density inner layer and a less dense outer layer.

FIG. 21 illustrates an example echogenic suture that plies together a strand of ePTFE with an echogenic strand or yarn.

FIG. 22 illustrates an example echogenic suture that includes an ePTFE core covered by an echogenic strand or yarn.

FIG. 23 illustrates a flowchart of an example method for repairing a cardiac valve using any of the echogenic sutures disclosed herein.

### DETAILED DESCRIPTION OF SOME EXAMPLES

The headings provided herein, if any, are for convenience only and do not necessarily affect the scope or meaning of the disclosed methods, systems, or devices.

### Overview

Doctors can perform a wide range of surgical procedures on a defective heart valve. In degenerative mitral valve repair procedures, techniques include, for example and without limitation, various forms of re-sectional repair, chordal implantation, ventricular reshaping, and edge-to-edge repairs. Clefts or perforations in a leaflet can be closed and occasionally the commissures of the valve sutured to minimize or eliminate MR. In these and similar procedures, ePTFE cords are typically used for performing the repairs, e.g., as artificial cords and/or sutures. For example, the mitral valve can be repaired by inserting ePTFE cords into the mitral valve and anchoring the cords to the ventricle or papillary muscle. Being able to visualize the sutures or cords during such procedures would be beneficial. For example, increasing suture visibility during beating heart mitral valve repair procedures can improve medical outcomes. Enabling the physician to more precisely determine a location of artificial cords and tension applied is highly advantageous. In addition, post mitral valve repair ultrasound follow-up visits would provide more accurate information. For example, it may be particularly difficult for an echocardiologist to determine if mitral regurgitation (MR) has increased due to cord pullouts, cord ruptures, or native cord ruptures. Accordingly, there is a need for sutures or cords with amplified echogenic signatures to increase visibility of the sutures using ultrasound technology.

To address these and other issues, disclosed herein are sutures (or cords) and methods for using sutures wherein the sutures comprise a material or a combination of materials configured to increase echogenicity of the sutures. In some examples, the sutures comprise echogenic materials incorporated into suture material (e.g., ePTFE). In some examples, the sutures comprise a combination of materials with abrupt and relatively large changes in density to increase reflections of ultrasound waves. The disclosed sutures enhance echogenicity to increase visibility of the sutures using ultrasound imaging technologies. Furthermore, disclosed herein are methods that utilize the disclosed sutures in valve repairs including implanting artificial cords, sub-valvular techniques, reshaping organs, annuloplasty, and the like. Utilization of the disclosed echogenic sutures can result in superior outcomes due at least in part to physicians being able to better visualize the placement and/or tension of the echogenic sutures.

The use of the disclosed echogenic sutures in the vascular system may provide a number of advantages. The disclosed echogenic sutures and associated methods may be advantageous due at least in part to their increased visibility using ultrasound imaging to provide real-time feedback during a procedure. In addition, the disclosed echogenic sutures may also improve the efficacy of follow-up visits due at least in part to clinicians being better able to see the condition of the implanted sutures. There are several cardiac procedures that may benefit from improved visualization due to the use of echogenic sutures including, for example and without limitation, cinching cords for annuloplasty, reshaping and annular reduction of the mitral valve, ventricular volume reduction and remodeling of dilated heart muscle, fixation of implantable cardiac devices, ultrasound detectable markers of a graft site, and the like. The disclosed echogenic sutures can be configured for use in the vascular system, or within the blood stream, and can be coated with a material to improve biostability and/or biocompatibility.

In some examples, in addition to valve repairs, the disclosed sutures may be advantageous in annuloplasty procedures, sub-valvular procedures, and procedures that are used to re-shape or modify the chambers of the heart and/or other internal organs. For example, the disclosed sutures may be used to pull the ventricle inwards to reduce the volume of the ventricle. The disclosed echogenic cords can be used in open heart procedures, less-invasive procedures, minimally invasive procedures, non-invasive procedures, transcatheter approaches, etc. Although principally described in the context of mitral valve repairs, it is to be understood that the disclosed cords can be used in tricuspid valve repairs and other valve repairs.

In some instances, disclosed methods for repairing tissue include inserting a delivery device, such as a delivery device described in the '761 PCT Application and/or in International Patent Application No. PCT/US2016/055170 (published as WO 2017/059426A1 and referred to herein as “the '170 PCT Application”), the entire disclosure of each of which is incorporated herein by reference, into a body and extending a distal end of the delivery device to a proximal side of the tissue. Advancement of the delivery device may be performed in conjunction with sonography or direct visualization (e.g., direct transblood visualization), and/or any other suitable remote visualization technique. Furthermore, one or more steps of the disclosed methods may also be performed in conjunction with any suitable remote visualization technique. With respect to the disclosed methods, one or more parts of a procedure may be monitored in conjunction with transesophageal (TEE) guidance or intracardiac echocardiography (ICE) guidance. For example, this may facilitate and direct the movement and proper positioning of the delivery device for contacting the appropriate target cardiac region and/or target cardiac tissue (e.g., a valve leaflet, a valve annulus, or any other suitable cardiac tissue). Typical procedures for use of echo guidance are set forth in Suematsu, Y., J. Thorac. Cardiovasc. Surg. 2005; 130:1348-56 (“Suematsu”), the entire disclosure of which is incorporated herein by reference.

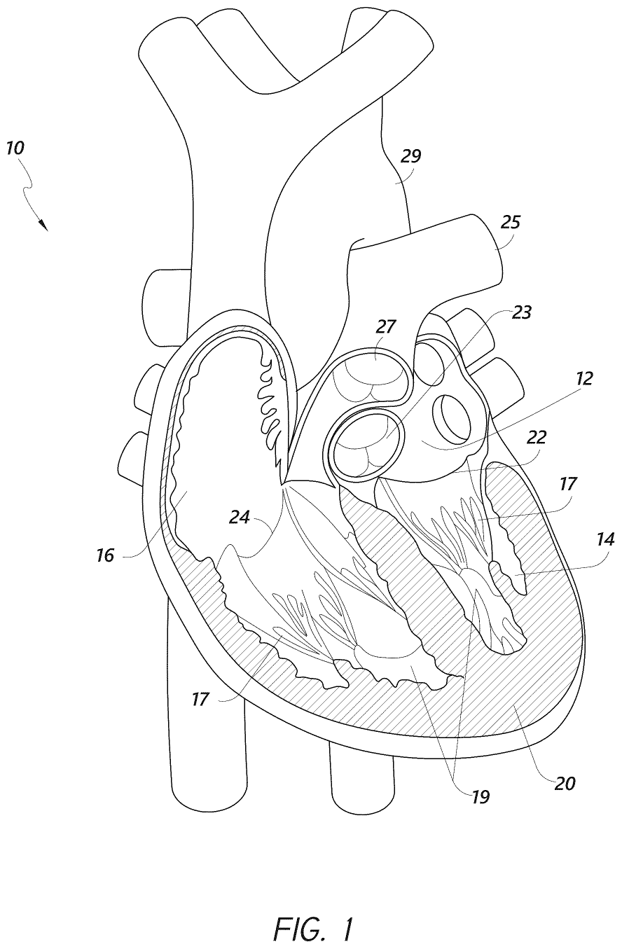

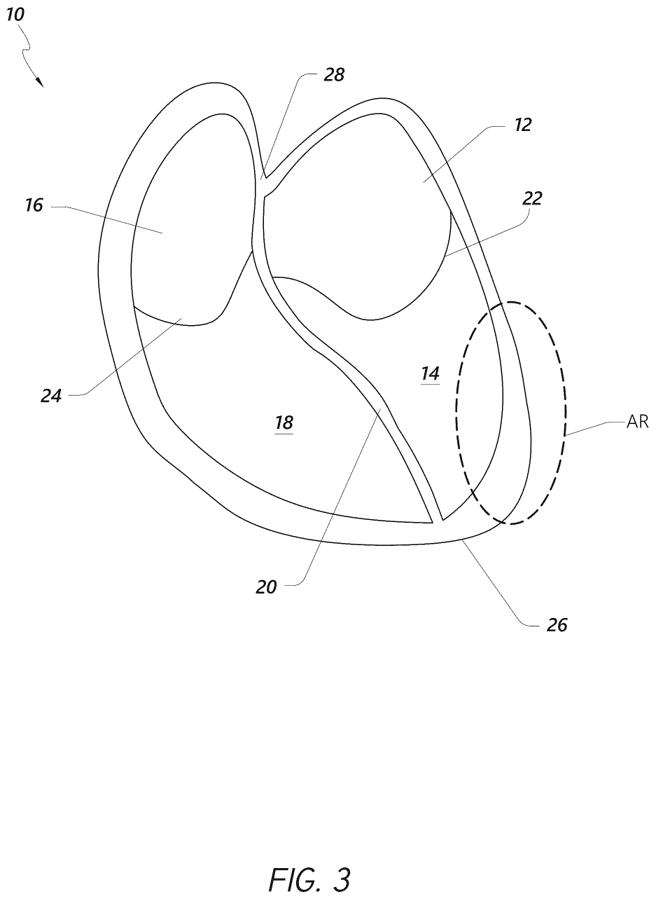

As illustrated in FIG. 1, the human heart 10 has four chambers, which include two upper chambers denoted as atria 12, 16 and two lower chambers denoted as ventricles 14, 18. A septum 20 (see, e.g., FIG. 3) divides the heart 10 and separates the left atrium 12 and left ventricle 14 from the right atrium 16 and right ventricle 18. The heart further contains four valves 22, 23, 26, and 27. The valves function to maintain the pressure and unidirectional flow of blood through the body and to prevent blood from leaking back into a chamber from which it has been pumped.

Two valves separate the atria 12, 16 from the ventricles 14, 18, denoted as atrioventricular valves. The mitral valve 22, also known as the left atrioventricular valve, controls the passage of oxygenated blood from the left atrium 12 to the left ventricle 14. A second valve, the aortic valve 23, separates the left ventricle 14 from the aortic artery (aorta) 29, which delivers oxygenated blood via the circulation to the entire body. The aortic valve 23 and mitral valve 22 are part of the “left” heart, which controls the flow of oxygen-rich blood from the lungs to the body. The right atrioventricular valve, the tricuspid valve 24, controls passage of deoxygenated blood into the right ventricle 18. A fourth valve, the pulmonary valve 27, separates the right ventricle 18 from the pulmonary artery 25. The right ventricle 18 pumps deoxygenated blood through the pulmonary artery 25 to the lungs wherein the blood is oxygenated and then delivered to the left atrium 12 via the pulmonary vein. Accordingly, the tricuspid valve 24 and pulmonic valve 27 are part of the right heart, which control the flow of oxygen-depleted blood from the body to the lungs.

Both the left and right ventricles 14, 18 constitute pumping chambers. The aortic valve 23 and pulmonic valve 27 lie between a pumping chamber (ventricle) and a major artery and control the flow of blood out of the ventricles and into the circulation. The aortic valve 23 and pulmonic valve 27 have three cusps, or leaflets, that open and close and thereby function to prevent blood from leaking back into the ventricles after being ejected into the lungs or aorta 29 for circulation.

Both the left and right atria 12, 16 are receiving chambers. The mitral valve 22 and tricuspid valve 24, therefore, lie between a receiving chamber (atrium) and a ventricle to control the flow of blood from the atria to the ventricles and prevent blood from leaking back into the atrium during ejection from the ventricle. Both the mitral valve 22 and tricuspid valve 24 include two or more cusps, or leaflets (not shown in FIG. 1), that are encircled by a variably dense fibrous ring of tissues known as the annulus (not shown in FIG. 1). The valves are anchored to the walls of the ventricles by chordae tendineae (chordae) 17. The chordae tendineae 17 are cord-like tendons that connect the papillary muscles 19 to the leaflets (not shown in FIG. 1) of the mitral valve 22 and tricuspid valve 24 of the heart 10. The papillary muscles 19 are located at the base of the chordae tendineae 17 and are within the walls of the ventricles. The papillary muscles 19 do not open or close the valves of the heart, which close passively in response to pressure gradients; rather, the papillary muscles 19 brace the valves against the high pressure needed to circulate the blood throughout the body. Together, the papillary muscles 19 and the chordae tendineae 17 are known as the sub-valvular apparatus. The function of the sub-valvular apparatus is to keep the valves from prolapsing into the atria when they close.

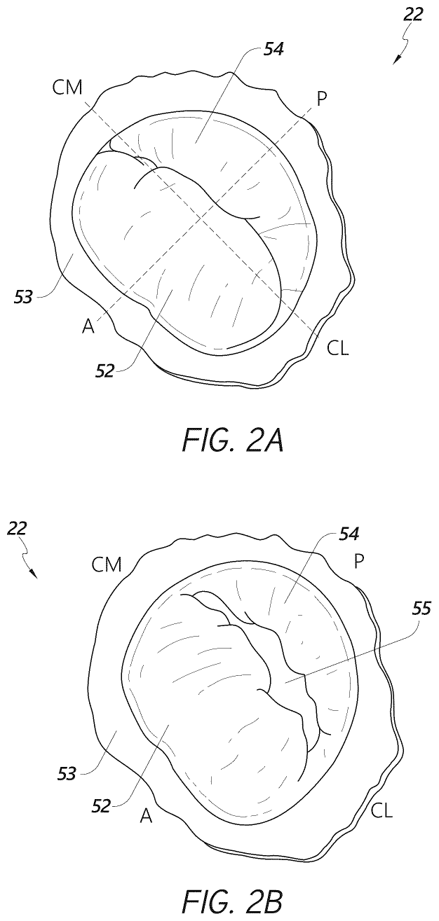

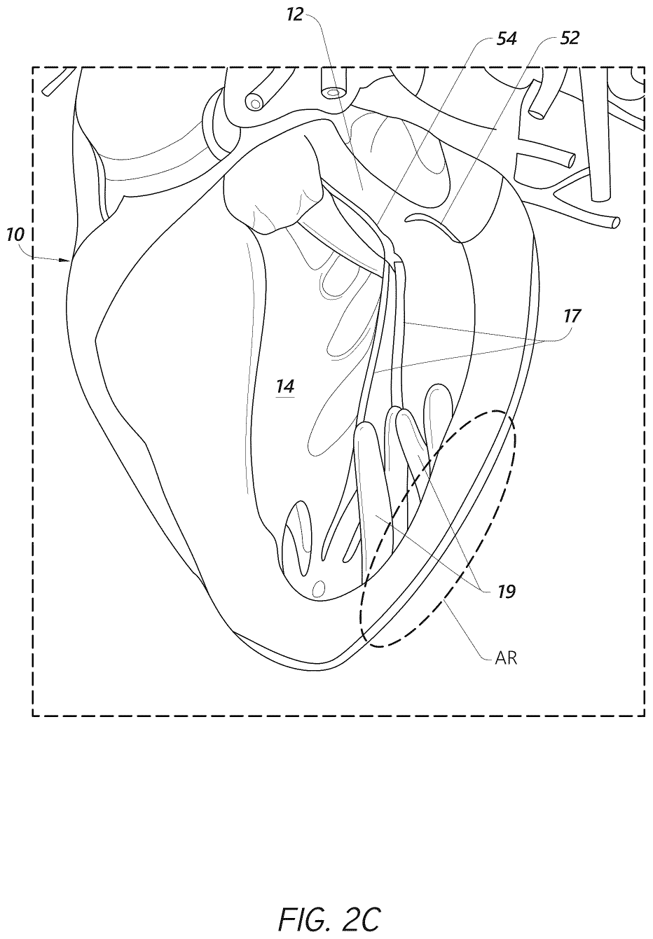

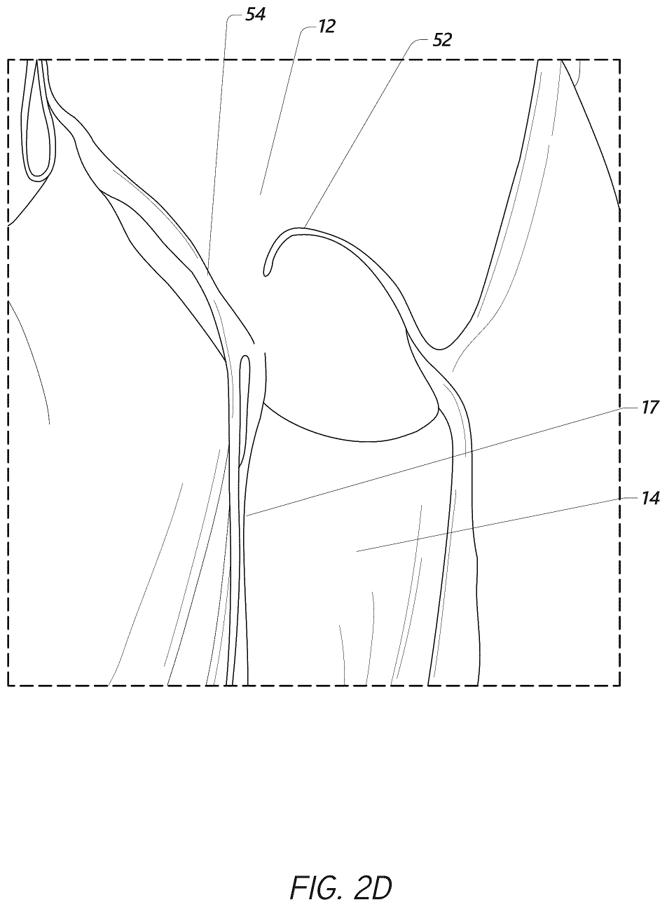

The mitral valve 22 is illustrated in FIG. 2A. The mitral valve 22 includes two leaflets, the anterior leaflet 52 and the posterior leaflet 54, and a diaphanous incomplete ring around the valve, called the annulus 53. The mitral valve 22 has two papillary muscles 19, the anteromedial and the posterolateral papillary muscles (see, e.g., FIG. 1), which attach the leaflets 52, 54 to the walls of the left ventricle 14 via the chordae tendineae 17 (see, e.g., FIG. 1).

FIG. 2B illustrates a prolapsed mitral valve 22. As can be seen with reference to FIGS. 2B-2D, prolapse occurs when a prolapsed segment of a leaflet 52, 54 of the mitral valve 22 is displaced above the plane of the mitral annulus into the left atrium 12 (see FIGS. 2C and 2D) preventing the leaflets from properly sealing together to form the natural plane or line of coaptation between the valve leaflets during systole. Because one or more of the leaflets 52, 54 malfunctions, the mitral valve 22 does not close properly, and, therefore, the leaflets 52, 54 fail to coapt. This failure to coapt causes a gap 55 between the leaflets 52, 54 that allows blood to flow back into the left atrium, during systole, while it is being ejected by the left ventricle. As set forth above, there are several different ways a leaflet may malfunction, which can thereby lead to regurgitation.

Mitral valve regurgitation increases the workload on the heart and may lead to very serious conditions if left untreated, such as decreased ventricular function, pulmonary hypertension, congestive heart failure, permanent heart damage, cardiac arrest, and ultimately death. Since the left heart is primarily responsible for circulating the flow of blood throughout the body, malfunction of the mitral valve 22 is particularly problematic and often life threatening.

Disclosed herein are echogenic sutures suitable for use in procedures to repair a cardiac valve, such as a mitral valve. Such procedures include procedures to repair regurgitation that occurs when the leaflets of the mitral valve do not coapt at peak contraction pressures, resulting in an undesired back flow of blood from the ventricle into the atrium. As described in the '761 PCT Application and the '170 PCT Application, after the malfunctioning cardiac valve has been assessed and the source of the malfunction verified, a corrective procedure can be performed. Various procedures can be performed to effectuate a cardiac valve repair, which will depend on the specific abnormality and the tissues involved. The procedures can include open heart procedures, less-invasive procedures, minimally invasive procedures, non-invasive procedures, procedures employing a transcatheter approach, etc. The procedures can include, for example and without limitation, implantation of artificial cords, annuloplasty, sub-valvular techniques (e.g., manipulating elements of the sub-valvular apparatus), reshaping of the heart, and the like.

FIG. 3 illustrates that one or more chambers, e.g., the left atrium 12, left ventricle 14, right atrium 16, or right ventricle 18, in the heart 10 may be accessed in accordance with any suitable method including open heart procedures, less-invasive procedures, minimally invasive procedures, non-invasive procedures, transcatheter approaches, etc. Access into a chamber 12, 14, 16, 18 in the heart 10 may be made at any suitable site of entry. In some examples, less-invasive procedures and non-invasive procedures can preferably gain access to the desired chamber of the heart through the apex region of the heart, for example, slightly above the apex 26 at the level of the papillary muscles 19 (see also FIG. 2C). Typically, access into the left ventricle 14 (e.g., to perform a mitral valve repair) is gained through the apical region, close to (or slightly skewed toward the left of) the median axis 28 of the heart 10. Typically, access into the right ventricle 18 (e.g., to perform a tricuspid valve repair) is gained through the apical region, close to or slightly skewed toward the right of the median axis 28 of the heart 10. Generally, an apex region of the heart is a bottom region of the heart that is within the left or right ventricular region and is below the mitral valve 22 and tricuspid valve 24 and toward the tip or apex 26 of the heart 10. More specifically, an apex region AR of the heart (see, e.g., FIG. 3) is within a few centimeters to the right or to the left of the septum 20 of the heart 10 at or near the level of the papillary muscles 19. Accordingly, the ventricle can be accessed directly via the apex 26, or via an off-apex location that is in the apical or apex region AR, but slightly removed from the apex 26, such as via a lateral ventricular wall, a region between the apex 26 and the base of a papillary muscle 19, or even directly at the base of a papillary muscle 19 or above.

The mitral valve 22 and tricuspid valve 24 can be divided into three parts: an annulus (see 53 in FIGS. 2A and 2B), leaflets (see 52, 54 in FIGS. 2A and 2B), and a sub-valvular apparatus. The sub-valvular apparatus includes the papillary muscles 19 (see FIG. 1) and the chordae tendineae 17 (see FIG. 1), which can elongate and/or rupture. If the valve is functioning properly, when closed, the free margins or edges of the leaflets come together and form a tight junction, the are of which, in the mitral valve, is known as the line, plane or area of coaptation. Normal mitral and tricuspid valves open when the ventricles relax allowing blood from the atrium to fill the decompressed ventricle. When the ventricle contracts, chordae tendineae properly position the valve leaflets such that the increase in pressure within the ventricle causes the valve to close, thereby preventing blood from leaking into the atrium and assuring that all of the blood leaving the ventricle is ejected through the aortic valve (not shown) and pulmonic valve (not shown) into the arteries of the body. Accordingly, proper function of the valves depends on a complex interplay between the annulus, leaflets, and sub-valvular apparatus. Lesions in any of these components can cause the valve to dysfunction and thereby lead to valve regurgitation. As set forth herein, regurgitation occurs when the leaflets do not coapt properly at peak contraction pressures. As a result, an undesired back flow of blood from the ventricle into the atrium occurs.

Although the procedures described herein are with reference to repairing a cardiac mitral valve or tricuspid valve using artificial cords, the cords and methods presented are readily adaptable for various types of tissue, leaflet, and annular repair procedures. In general, the methods herein are described with reference to a mitral valve 22 but should not be understood to be limited to procedures involving the mitral valve.

### Examples of Cardiac Repairs Using Echogenic Sutures

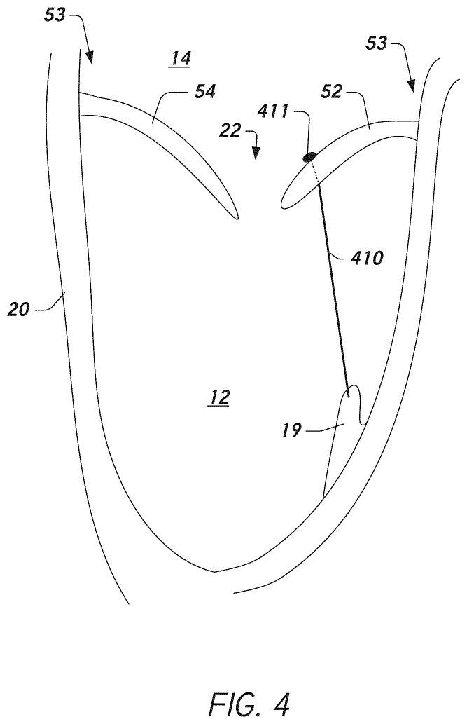

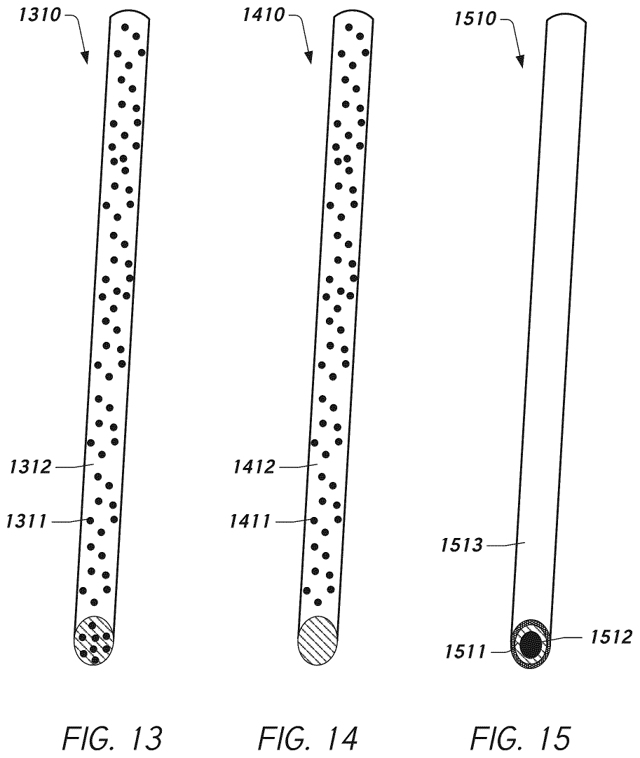

FIG. 4 illustrates the use of one or more echogenic cords 410 to repair a mitral valve 22. Although described with respect to repairing the mitral valve, the disclosed cords can be used to repair a tricuspid valve or other cardiac valve. The one or more echogenic cords 410 are echogenic sutures or cords, examples of which are disclosed in greater detail herein with respect to FIGS. 13-22. The mitral valve 22 can be repaired by inserting the echogenic cords 410 into the mitral valve 22 and anchoring the echogenic cords 410 to the ventricle 12 and/or papillary muscle 19. In some examples, the echogenic cords 410 can be anchored to the septum 20. The echogenic cords 410 can be attached to the anterior leaflet 52 using a distal anchor 411. In some examples, the echogenic cords 410 can be attached to the posterior leaflet 54 and/or the annulus 53. The distal anchor 411 can be any suitable anchor including, for example, hooks, barbs, knots, grafts, fabric, etc., or any combination thereof. The distal anchor 411 can be formed of any suitable material. In some instances, for example, the material of the distal anchor 411 can be the same as the echogenic cords 410 or can be any one or more of ePTFE sutures, polybutylate-coated polyester sutures, or polyester sutures (such as, for example, ETHIBOND EXCEL® polyester sutures).

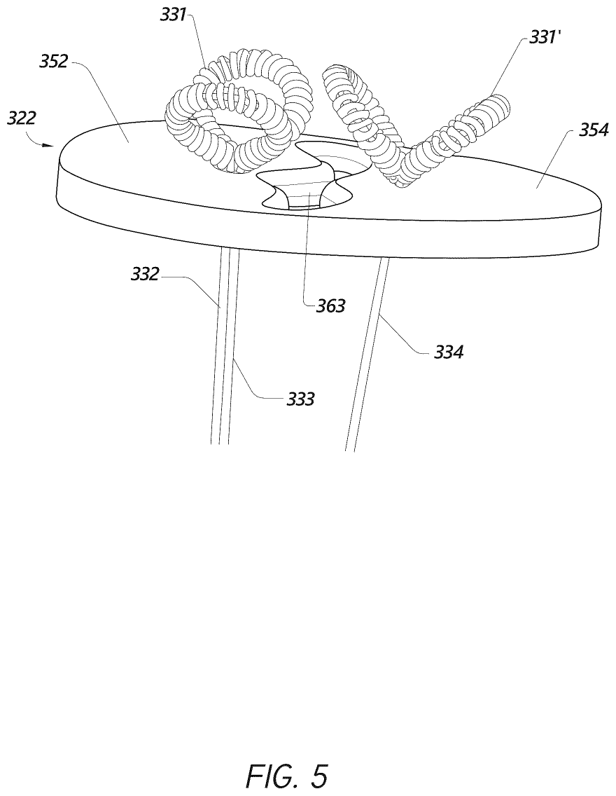

In some examples, the distal anchor 411 can be made from a distal portion of the echogenic cords 410, e.g., a bulky knot, an example of which is described herein with respect to FIG. 5. In some examples, the distal portion of the echogenic cords 410 can be secured to the annulus 53 and/or the posterior leaflet 54 in addition to or as an alternative to the anterior leaflet 52.

The echogenic cords 410 can be any of the echogenic cords described herein. For example, the echogenic cords 410 can comprise echogenic sutures that have a tailored topology, that include enhancing materials, and/or that have acute changes in density within the suture. Examples of echogenic sutures are described herein with reference to FIGS. 13-22. Advantageously, using the echogenic cords 410 improves visualization of the artificial cords during and after the procedure. This enhanced visualization can be used to adjust a position or tension of the echogenic cords 410 during the procedure, to detect tangles or knots in the suture during or after the procedure, and to detect ruptures or other failures in the suture after the procedure.

FIG. 5 is a schematic illustration of a mitral valve 322 with leaflets 352, 354 that are separated by a gap 363. Two anchors 331, 331′ (e.g., bulky knot implants) are disposed on an atrial, distal, or top side of the leaflets 352, 354, respectively. Sutures 332, 333, 334 extend proximally from the anchors 331, 331′ (e.g., the bulky knot implants). The sutures 332, 333, 334 are echogenic sutures, as described herein.

The anchors 331, 331′ can be formed with the same suture material as the sutures 332, 333, 334. The suture material for the anchors 331, 331′ forms one or more loops on the atrial side of the leaflets 352, 354 and extends through the leaflets 352, 354, with sutures 332, 333, 334, that can include two loose suture end portions, that extend on the ventricular, proximal, or bottom side of the leaflets 352, 354. The anchor 331 has sutures 332 and 333, and the anchor 331′ has suture 334 (and another end portion not shown in FIG. 5). The suture material forming the anchors 331, 331′ can be braided, twisted, or knotted (e.g., with overhand knots) to form the anchors 331, 331′.

After the anchors 331, 331′ are in a desired or targeted position (which can be confirmed with imaging, for example), a device can be used to secure the anchors 331, 331′ in the desired position and to secure the leaflets 352, 354 of the valve in an edge-to-edge relationship. Further, in addition to or instead of creating the edge-to-edge relationship, to promote a larger surface of coaptation, the anchors 331, 331′ can be secured together to pull or otherwise move the posterior annulus towards the anterior leaflet and/or the anterior annulus towards the posterior leaflet, to reduce the distance between the anterior annulus and the posterior annulus, e.g., the septal-lateral distance by about 10%-40%. Approximating the anterior annulus and the posterior annulus in this manner can decrease the valve orifice, and thereby decrease, limit, or otherwise prevent undesirable regurgitation.

Examples of forming distal anchors, pre-formed knots, and/or locking sutures are presented in U.S. Pat. No. 8,852,213, International Patent Publication No. 2017/059426, and U.S. Patent Publication No. 2019/0117401, each of which is incorporated by reference herein in its entirety for all purposes. For each of these anchors and/or knots, the material(s) used can be the same as the material(s) used for the sutures 332, 333, 334, e.g., echogenic sutures.

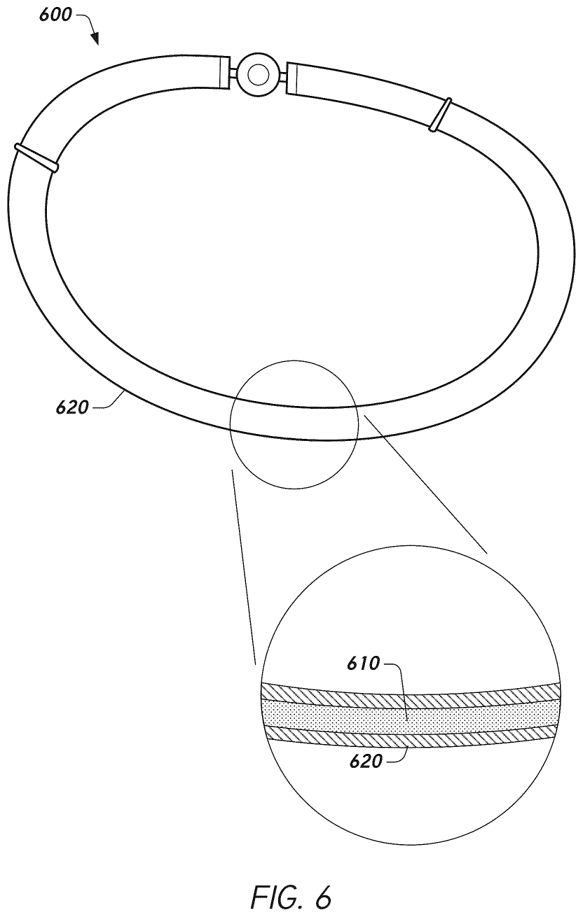

FIG. 6 illustrates an example of an annuloplasty ring 600 that has a core 610 surrounded by a jacket 620, the core 610 comprising any of the echogenic sutures disclosed herein. The annuloplasty ring 600 can be used to reshape, reinforce, or tighten the annulus around a heart valve. The annuloplasty ring 600 can be used in a procedure by itself or it can be used in conjunction with other procedures, such as the procedures described herein, to repair a cardiac valve.

The jacket 620 can be made of any suitable combination of durable plastic, metal, and fabric. Suitable materials include, for example and without limitation, silicone rubber, polyester knit fabric, plastic strips, titanium alloys, siloxane polymer rubber, non-magnetic cobalt-chromium-nickel-molybdenum alloy, etc. The jacket 620 can be configured so that the annuloplasty ring 600 has variable flexibility. For example, variable flexibility of the annuloplasty ring 600 allows for physiologic contractility of the valve in which it is implanted during systole. The core 610 can include any of the echogenic sutures disclosed herein. The core 610 can be a suture with echogenic material and/or with materials that provide acute changes in density. The echogenic sutures can enhance visualization of the annuloplasty ring 600. In some examples, the core 610 can be used to adjust the size, shape, tension, etc. of the annuloplasty ring 600 to improve performance of the valve after implantation of the annuloplasty ring 600.

FIG. 7 illustrates an example sub-valvular procedure using one or more echogenic sutures 710. The echogenic sutures 710 can be any of the echogenic sutures described herein with reference to FIGS. 13-22. Sub-valvular procedures include procedures to reposition one or more papillary muscles 19 or manipulation or alteration of one or more chordae tendineae 17 to improve valve function (e.g., to reduce regurgitation).

The illustrated sub-valvular procedure anchors the one or more echogenic sutures 710 to the papillary muscle 19 and to a wall of the heart to relocate the papillary muscle 19 to improve performance of the chordae tendineae 17. The one or more echogenic sutures 710 can be anchored to additional papillary muscles 19 of the anterior leaflet 52 and/or to papillary muscles of the posterior leaflet 54. In some examples, an echogenic suture 710 can be used to form a loop around multiple papillary muscles to approximate the papillary muscles. In certain examples, an echogenic suture 710 can be anchored to another part of the anatomy such as the atrium 14, the annulus 53, or outside the heart.

The illustrated procedure may be generally referred to as papillary muscle relocation. Other similar sub-valvular procedures may also utilize the one or more echogenic sutures 710. Example sub-valvular procedures that may use the disclosed echogenic sutures 710 include, for example and without limitation, papillary muscle relocation, papillary muscle sling, papillary muscle approximation, papillary muscle sandwich plasty, “ring and string” procedures, or ring noose string. In some examples, these procedures are performed in conjunction with implantation of an annuloplasty ring, as disclosed herein.

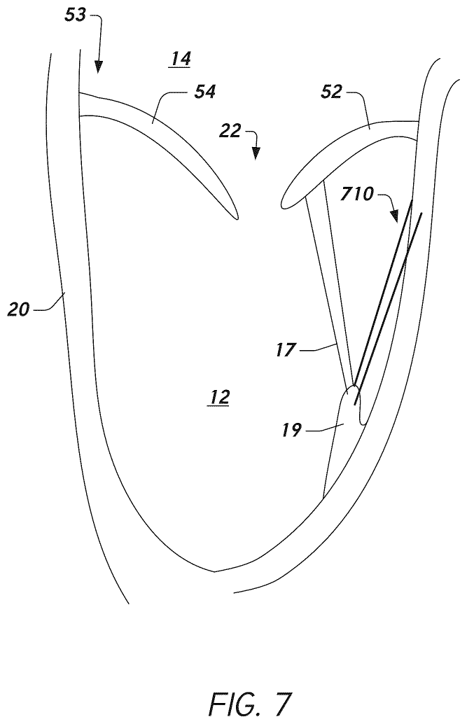

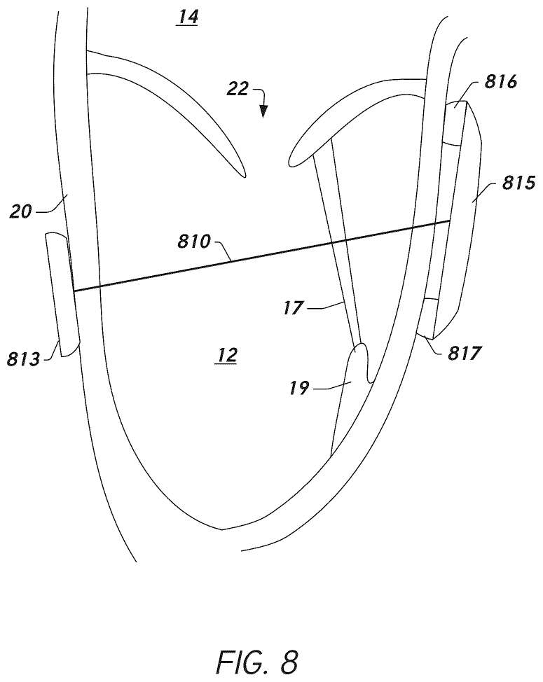

FIG. 8 illustrates using an echogenic suture 810 in a procedure to reshape a portion of the heart. The echogenic suture 810 can be any of the echogenic sutures described herein with reference to FIGS. 13-22. To reshape the heart, an anterior pad 813 can be implanted or anchored to an epicardial surface of the heart on one side of a targeted chamber of the heart (e.g., the ventricle 12). A posterior pad 815 can be implanted or anchored to the heart on another side of the targeted chamber. The echogenic suture 810 extends between the anterior pad 813 and the posterior pad 815. The length of the echogenic suture 810 can be adjusted to approximate the anterior pad 813 and the posterior pad 815 to reshape the targeted chamber. In some examples, the anterior pad 813 is adjustable and is fixed to the echogenic suture 810 after sizing the device. In some examples, the posterior pad 815 has a superior head 816 and an inferior head 817 configured to change a shape at the level of the annulus and the level of the papillary muscle, respectively.

In some examples, the transventricular chordal length can be reduced by about 25%. This procedure advantageously can affect and stabilize both the mitral annulus and papillary muscles, can be implanted in an off-pump procedure, can be easily reversed, and has little or no effect on annular dynamics.

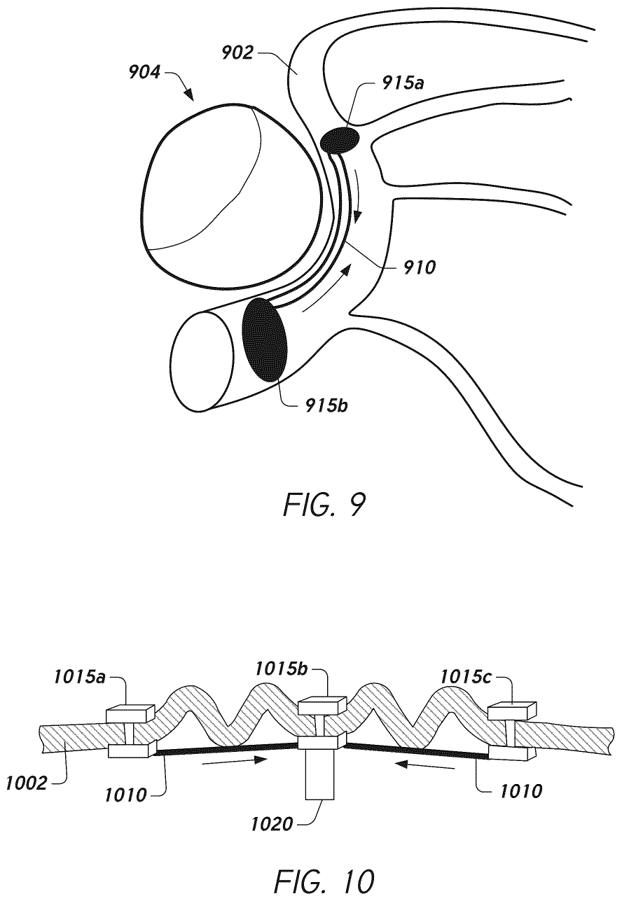

FIG. 9 illustrates using an echogenic suture 910 in an annuloplasty procedure that implants the echogenic suture 910 in the coronary sinus 902 to reshape the annulus 904 of the mitral valve. The procedure involves implanting a pair of anchors 915a, 915b in the coronary sinus 902, the pair of anchors connected by an echogenic suture 910, and then cinching or tightening the echogenic suture 910 to reduce the distance between the anchors 915a, 915b, thereby reducing the size and/or altering the shape of the annulus 904 of the mitral valve. This procedure thus uses an intravascular support that is designed to change the shape of the annulus that is adjacent to the coronary sinus in which the support is placed. The support is designed to aid the closure of a mitral valve. The support is placed in the coronary sinus and vessel that are located adjacent the mitral valve and urges the vessel wall against the valve to aid its closure.

FIG. 10 illustrates using an echogenic suture 1010 in an annuloplasty procedure that implants a plurality of anchors 1015a, 1015b, 1015c in an annulus 1002 of a mitral valve and pulls the anchors 1015a-1015c together using the echogenic suture 1010 to reshape the annulus 1002. In some examples, pledgeted anchors 1015a-1015c are deployed on the posterior mitral annulus at P1P2 and P2P3 locations. These anchors 1015a-1015c are cinched to reduce the annulus 1002 using the echogenic suture 1010. The procedure includes anchoring tissue with anchor assemblies 1015a-1015c comprising a proximal end portion, a distal end portion and a compressible intermediate portion located between the proximal and distal end portions and movable between an elongated configuration and a shortened configuration. The procedure includes inserting at least one of the anchor elements 1015a-1015c through the tissue of the annulus 1002 and pulling the echogenic suture 1010 relative to the other anchor elements. This draws the proximal and distal end portions of the anchor assembly toward each other and compresses the intermediate portion into the shortened configuration with the assembly engaged against the tissue. The tissue may comprise the mitral valve annulus and the anchor assemblies may be engaged on opposite sides of the tissue, such as on opposite sides of the mitral valve annulus. The procedure includes drawing the anchor assemblies toward each other to plicate the tissue of the annulus 1002 whereupon the anchor assemblies are locked relative to each other to lock the plicated condition of the tissue of the annulus 1002. This procedure may, for example, be repeated any number of times to plicate the posterior portion of the mitral valve annulus for purposes of achieving annuloplasty. A mechanism 1020 can be used to cinch the echogenic suture to draw the anchor assemblies toward each other.

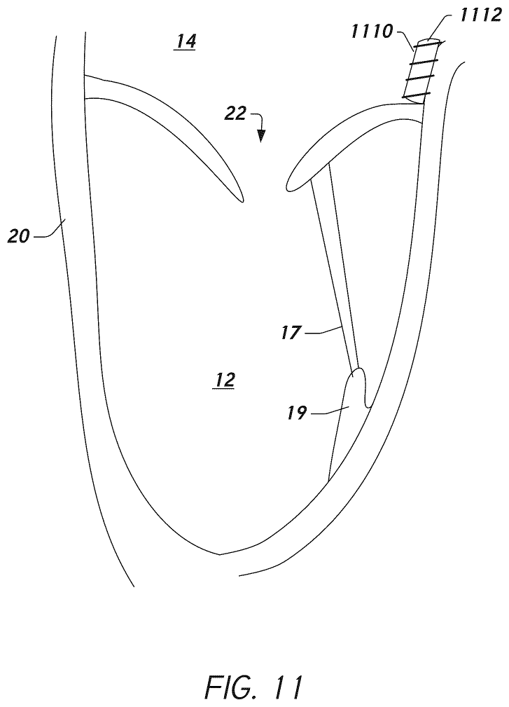

FIG. 11 illustrates using an echogenic suture 1110 for the fixation of a cardiac device 1112 in the heart. The echogenic suture 1110 can be used to implant the cardiac device 1112. The cardiac device 1112 can be any suitable device including a stent, a sensor, a flow controller, a pacemaker, or the like. The echogenic suture 1110 can facilitate visualizing the process of implanting the cardiac device 1112 and in checking on the condition of the implantation of the cardiac device 1112 in follow up visits.

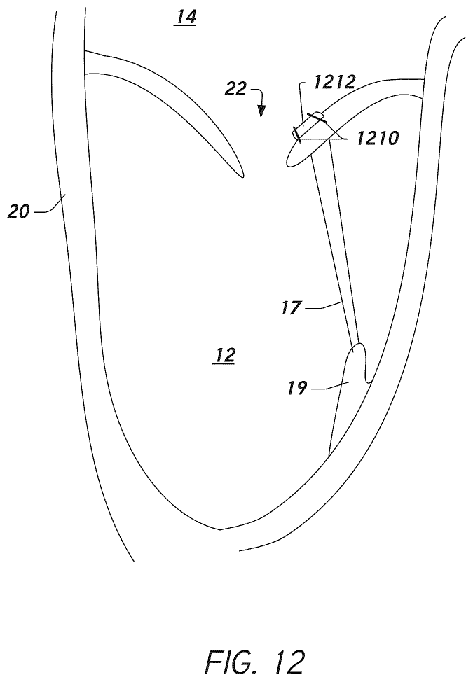

FIG. 12 illustrates using an echogenic suture 1210 as an ultrasound detectable marker of a graft site. The echogenic suture 1210 can mark a graft site and/or can be used to secure a graft 1212 to tissue. The echogenic suture 1210 can mark the location of a graft, facilitate visualizing the process of implanting the graft 1212, and aid in checking on the condition of the implantation of the graft 1212 in follow up visits.

In some examples, an echogenic suture can be used to close perforations in the ventricular or atrial septum.

### Examples of Echogenic Sutures

Increasing the echogenicity of a suture can include altering the composition of the suture. This can be achieved by changing the suture topology and/or the density of one or more materials. There are a variety of suitable echogenic enhancing materials that can be used to increase the echogenicity of a suture to create a highly-reflective object that can be readily imaged using ultrasound. These materials include, for example and without limitation, microbubbles (e.g., a gas such as C₃F₈ surrounded by a lipid shell), silica nanoparticles, hollow glass microspheres, glass beads, and echogenic biomaterials.

There are variety of methods of incorporating an echogenic material into a suture. For example, the echogenic material can be incorporated directly on the suture with the use of a pre-coat polymer bonding layer to promote adhesion of the echogenic material. As another example, the echogenic material can be mixed with a solvent and applied using standard coating methods, such as by spraying, dipping, roll coating, bar coating, spin coating, or a wiping process. As another example, the echogenic material can be compounded into the resin and subsequently extruded. As another example, the echogenic material can be mechanical trapped between layers. As another example, strands of the echogenic material can be twisted, plied, covered, interlaced, interlooped, or intertwined between layers using a textile preparation process.

The general structure of the echogenic sutures described herein may be a single polymer with an echogenic material dispersed throughout, or the suture can be made of two or more materials. Where more than one material is used, the echogenic material can be applied to one or the materials to tailor the echogenic signature. This may also be of benefit as it limits the impact the echogenic material has on the mechanical properties of the suture.

FIG. 13 illustrates an example echogenic suture 1310 that includes echogenic material 1311 embedded throughout a polymer 1312. In some examples, the echogenic material 1311 can be added to the polymer by extruding the echogenic material, such as glass beads, into a rod of polymer. Typically for cardiac repairs, ePTFE material is used for the sutures or cords. A benefit of using ePTFE material is that it promotes the formation of endothelial tissue due at least in part to its relatively high porosity. This is beneficial because endothelialization strengthens the ePTFE cords. Thus, the polymer 1312 may be ePTFE or other suitable polymer such as ultra-high-molecular-weight polyethylene (UHMWPE), polyethylene terephthalate (PET), polyether ether ketone (PEEK), polypropylene (PP), ePTFE, PTFE, or polystyrene (PS). In addition, the echogenic material 1311 can be any suitable echogenic material described herein.

FIG. 14 illustrates an example echogenic suture 1410 that includes echogenic material 1411 embedded on a surface of a polymer 1412. The echogenic material 1411 can be applied to a surface of the polymer 1412 using any of the techniques described herein, such as spraying, dipping, roll coating, bar coating, spin coating, or a wiping process. The polymer 1412 can be any suitable polymer described herein. In addition, the echogenic material 1411 can be any suitable echogenic material described herein.

FIG. 15 illustrates an example echogenic suture 1510 that includes an inner polymer layer 1512 that is coated with a layer of echogenic material 1511 that is surrounded by an outer polymer layer 1513. In some examples, the echogenic material 1511 can be embedded into a coating and the coating can be applied to the inner polymer layer 1512. The inner polymer layer 1512 and the outer polymer layer 1513 can be any suitable polymer described herein and both layers can be the same polymer or different polymers. In addition, the echogenic material 1511 can be any suitable echogenic material described herein.

A variety of coating processes can be used to apply the echogenic material 1511 to the inner polymer layer 1512 and/or the outer polymer layer 1513 to the echogenic material 1511 such as braiding, wrapping, extruding, laminating, dipping, spraying, or spin coating the material around the layer it is surrounding. By surrounding the echogenic material 1511 with the outer polymer layer 1513, the benefits of using the polymer (e.g., ePTFE material) can be realized while also increasing the echogenicity of the echogenic suture 1510. For example, the outer polymer layer 1513 can promote tissue growth or endothelialization, as described herein. This may improve or enhance the strength of the echogenic suture 1510. In addition, the outer polymer layer 1513 can have a relatively low coefficient of friction thereby reducing wear.

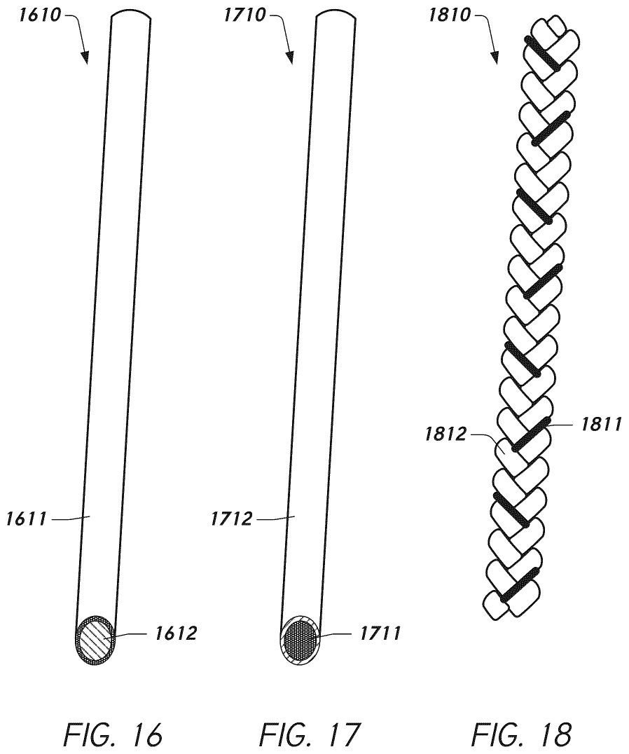

FIG. 16 illustrates an example echogenic suture 1610 that includes a polymer layer 1612 fully coated by echogenic material 1611. This can be accomplished using any of the procedures described herein. The polymer layer 1612 can be any suitable polymer described herein. In addition, the echogenic material 1611 can be any suitable echogenic material described herein.

FIG. 17 illustrates an example echogenic suture 1710 that includes an echogenic material 1711 surrounded by a polymer layer 1712. The polymer layer 1712 can be configured to improve biostability and/or biocompatibility. Examples of such polymers include, for example and without limitation, hydrophilic coatings, hydrophobic coatings, or polymer sleeves or coatings (e.g., UHMWPE, PET, PEEK, PP, or PS). In addition, the echogenic material 1711 can be any suitable echogenic material described herein.

FIG. 18 illustrates an example echogenic suture 1810 that includes a strand of echogenic material 1811 weaved between braids 1812 of the echogenic suture 1810. The braids 1812 can be a bundle or assembly of individual filaments, sometimes referred to as a yarn. Each braid 1812 in the echogenic suture 1810 can be a polymer filament. The echogenic material 1811 can include a filament that has been modified to include echogenic material, as described herein. The braids 1812 can be any suitable polymer described herein. In addition, the echogenic material 1811 can be any suitable echogenic material described herein.

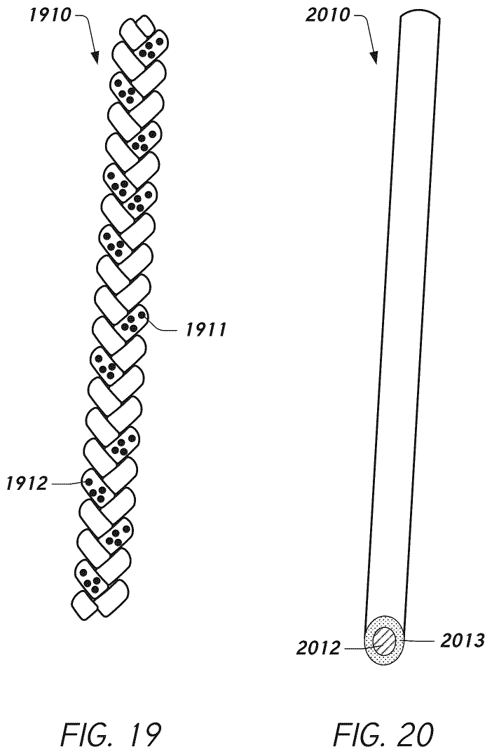

FIG. 19 illustrates an example echogenic suture 1910 that includes an echogenic material 1911 embedded between braids 1912 of the echogenic suture 1910. In some examples, the echogenic material 1911 can be embedded between braids 1912 of the echogenic suture 1910 by depositing echogenic material onto the material of the braids 1912 and then braiding the material with the deposited echogenic material to form the braids 1912, thereby trapping the echogenic material between weaves. The braids 1912 can be any suitable polymer described herein. In addition, the echogenic material 1911 can be any suitable echogenic material described herein.

The braids of FIGS. 18 and 19 can be configured to mimic the porosity of ePTFE cords. This may be beneficial to promote endothelialization, where desirable. The braided or twisted strands can promote tissue growth between the strands. The braided strands can form a tubular braid structure. This can be formed by crossing a number of strands of a desired material (e.g., polymers and echogenic material) diagonally in such a way that each group of strands pass alternately over and under a group of strands laid up in the opposite direction. The strands can have a variety of cross-sectional shapes including, but not limited to, round, square, rectangular, etc. The strands can be intertwined using three or more parallel strands of material. The strands can be interlaced in a variety of different patterns. These patterns influence the order of interlacing points in the braid structure and can affect the mechanical properties of the braid's structure. Areas in-between the braids can serve as a space for deposition and adhesion of endothelial cells. This added new layer of endothelialization, can be promising in improving durability. The use of braided sutures can increase the mechanical strength, structural integrity, and durability of a cardiac repair by providing sufficient support to withstand the increase chordal tension associated with high intracardiac pressures.

FIG. 20 illustrates an example echogenic suture 2010 that includes an acute density change between a higher density inner layer 2012 and a less dense outer layer 2013. The higher density inner layer 2012 and the less dense outer layer 2013 can be any suitable polymers that result in an acute change in density between the two layers. The acute change in density may also be described as an abrupt and relatively large change in density between the two layers. Rather than including an echogenic material, the echogenic suture 2010 relies on the acute density change to increase or promote reflections of ultrasound waves off of the echogenic suture 2010 to enhance echogenicity.

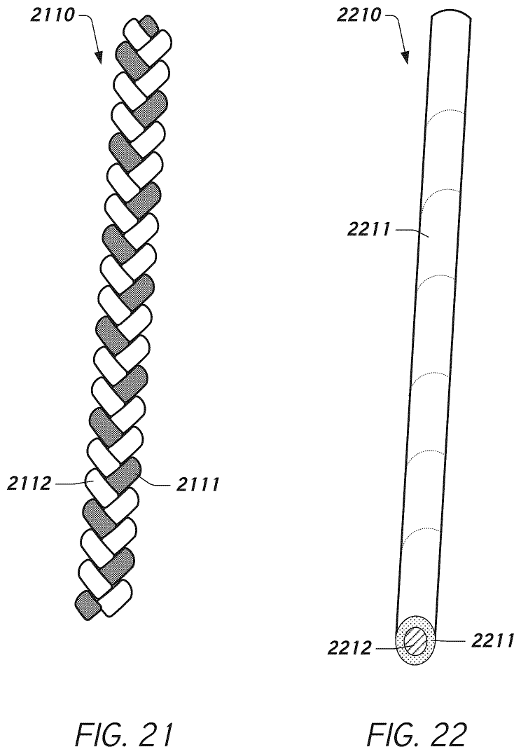

FIG. 21 illustrates an example echogenic suture 2110 that plies together an ePTFE strand 2112 with an echogenic strand 2111 or yarn. As described herein, the echogenic strand 2111 can be manufactured by embedding echogenic material onto or into another material, such as a polymer, using any suitable procedure such as extruding, coating, adhering, etc.

FIG. 22 illustrates an example echogenic suture 2210 that includes an ePTFE core 2212 covered by an echogenic sheath 2211. In some implementations, the echogenic sheath 2211 can be made of echogenic strands or yarn. The echogenic sheath 2211 can be formed to surround the ePTFE core 2212. The echogenic sheath 2211 can be formed from ribbons of flattened echogenic material wrapped around the ePTFE core 2212. In some examples, the echogenic material (e.g., a polymer with echogenic material incorporated therein) can be flattened into a ribbon, wrapped around the ePTFE core 2212, and heat fused to fuse the wrinkles together.

### Example Methods for Repairing Cardiac Valves with Echogenic Sutures

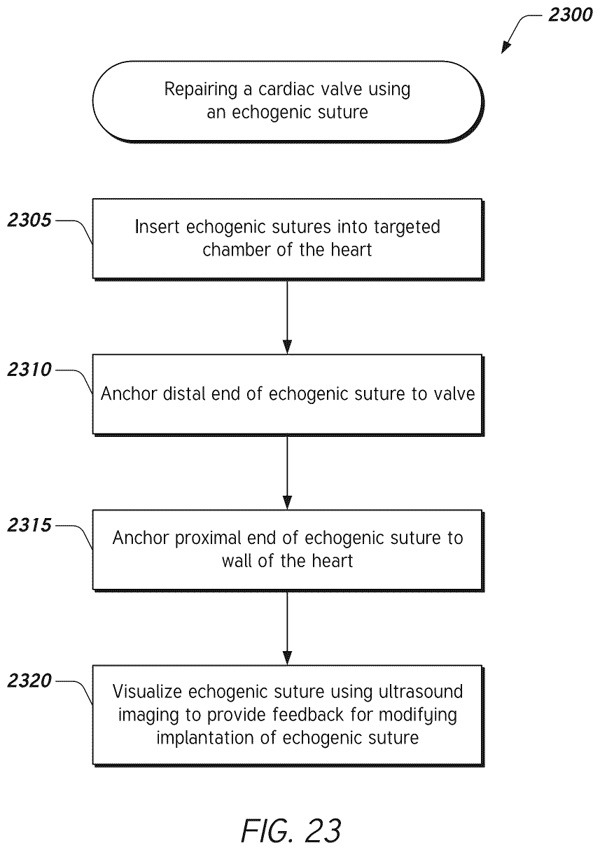

FIG. 23 illustrates a flowchart of an example method 2300 for repairing a cardiac valve (e.g., mitral valve, tricuspid valve, pulmonary valve, aortic valve) using any of the echogenic sutures disclosed herein. In block 2305, an echogenic suture is inserted into a targeted chamber (e.g., through a wall of the heart or using a transcatheter approach). For example, to repair the mitral valve, the echogenic suture can be inserted through the ventricle. The echogenic suture is any of the sutures or cords disclosed herein, including the sutures described herein with respect to FIGS. 13-22.

In some examples, a delivery device can be used to deliver the echogenic suture to the targeted chamber of the heart using a minimally invasive procedure. A piercing portion of the delivery device can be used to form an opening in the tissue, through which the distal end of the delivery device can be inserted. However, it is to be understood that any suitable procedure may be employed including an open-heart procedure, a less-invasive procedure, a non-invasive procedure, and/or a transcatheter approach.

In block 2310, a distal end of the echogenic suture is anchored to the tissue of the targeted valve. When repairing a mitral valve, for example, the tissue of the targeted valve can include the posterior leaflet, the anterior leaflet, and/or the annulus of the valve. The distal end of the echogenic suture can include a bulky knot as the anchor, examples of which are described herein with respect to FIG. 5. The distal end of the echogenic suture can include any suitable anchor for securing the echogenic suture to the tissue of the targeted valve, as described in greater detail herein.

The delivery device can be used to form or deliver a distal anchor to the distal side of the tissue of the targeted valve. The delivery device can be used in this manner to deliver two or more anchors to the distal side of the tissue. The anchors can be delivered to a single tissue (e.g., a posterior mitral valve leaflet), or one or more anchors can be delivered to a first tissue (e.g., a posterior mitral valve leaflet), and one or more other implants can be delivered to a second tissue (e.g., an anterior mitral valve leaflet, a mitral valve annulus, or any other suitable tissue) separate from the first tissue.

In block 2315, a proximal end of the echogenic suture is anchored to the wall of the heart and/or a papillary muscle of the heart. To anchor the proximal end of the echogenic cord, a pledget may be used.

In block 2320, the echogenic suture is visualized using ultrasound imaging. The visualization or imaging can be provided for feedback to monitor, to adjust, to fine tune, or to modify implantation of the echogenic suture. The visualization step in block 2320 can be performed concurrently with the steps in blocks 2305, 2310, and 2315.

The delivery device can then be withdrawn, and suture portions extending from the anchors can extend to a location (e.g., an outside surface of the heart or other suitable organ) remote from the tissue(s). The suture portions are the echogenic sutures that comprise features and/or materials to enhance echogenicity, as described herein. Where the term anchor is used herein, it is to be understood that an anchor refers to any suitable component or element that serves to anchor a suture to tissue such as, for example and without limitation, hooks, barbs, knots (e.g., bulky knots), and the like. In certain instances, the secured echogenic suture can be suitably tensioned and/or pulled towards the access site, e.g., into the ventricle of the heart, resulting in a larger effective surface area of coaptation and improved coaptation between the leaflets.

The anchoring step is done to prevent or to reduce the likelihood that the sutures come loose. The echogenic sutures can be anchored to a tissue wall, such as an external wall of the heart. A pledget can be used as the anchor. For example, PTFE (TEFLON®, Dupont, Wilmington, Delaware) felt can be used as an anchor where the felt is attached to the tissue wall. In some examples, the anchor includes holes through which the echogenic suture extends. Knots and/or locking sutures can be used to anchor the sutures.

In the methods disclosed herein, additional anchors and cords may be implanted. For example, to promote a larger surface of coaptation, anchors may be deployed in the body of the leaflets and/or at or near the annulus of the anterior and posterior leaflets, and the cords extending therefrom can be secured together and pulled to move the posterior annulus towards the anterior leaflet and/or the anterior annulus towards the posterior leaflet, thereby reducing the distance between the anterior annulus and the posterior annulus, e.g., the septal-lateral distance. Said another way, approximating the anterior annulus and the poster annulus in this manner can decrease the valve orifice, and thereby decreases, limits, or otherwise prevents undesirable regurgitation. One or more of the additional cords can be echogenic sutures and/or one or more of the additional cords can be of a different type (e.g., ePTFE sutures).

The method 2300 may be modified to reshape an internal organ rather than repairing a cardiac valve, an example of which is described herein with reference to FIG. 8. For example, the echogenic sutures can be anchored to enable the application of a force to move, shape, and/or remodel any part of an internal organ, such as the heart. The method 2300 may also be modified to include the implantation of an annuloplasty ring, an example of which is described herein with reference to FIG. 6. For example, the annuloplasty ring with an echogenic suture as a core material can be used to reduce the size of the annulus to improve performance of the valve (e.g., improve coaptation and/or to reduce regurgitation). Furthermore, the method 2300 may also be modified to manipulate papillary muscles to improve performance of the valve, generally referred to as sub-valvular techniques, an example of which is described herein with reference to FIG. 7. For example, an echogenic suture can be secured to one or more papillary muscles and then used to relocate or otherwise manipulate the one or more papillary muscles with the purpose of improving valve performance. Furthermore, the method 2300 may also be modified to perform an annuloplasty procedure to reshape the annulus, examples of which are described herein with reference to FIGS. 9-10. Furthermore, the method 2300 may also be modified to implant a device or a graft in the heart, examples of which are described herein with reference to FIGS. 11-12.

The above-described procedures can be performed manually, e.g., by a physician, or can alternatively be performed fully or in part with robotic or machine assistance. Further, although not specifically described herein, in various instances the heart may receive rapid pacing to reduce the relative motion of the edges of the valve leaflets during the procedures described herein (e.g., while an anchor, suture, and/or locking suture is being delivered and deployed).

### Additional Examples and Terminology

While various examples have been described above, it should be understood that they have been presented by way of illustration only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or examples described above indicate certain components arranged in certain orientations or positions, the arrangement of components may be modified. While the examples have been particularly shown and described, it will be understood that various changes in form and details may be made. Any portion of the apparatus and/or methods described herein may be combined in any combination, except mutually exclusive combinations. The examples described herein can include various combinations and/or sub-combinations of the functions, components and/or features of the different examples described.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings provided herein can be applied to other methods and systems and are not limited to the methods and systems described above, and elements and acts of the various examples described above can be combined to provide further examples. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. An echogenic suture for medical procedures wherein an echogenic signal is augmented by acute changes in material density of materials in the echogenic suture.

2. The echogenic suture of claim 1, wherein the materials of the echogenic suture comprise a polymer with echogenic material embedded therein.

3. The echogenic suture of claim 1, wherein materials of the echogenic suture comprise a polymer with echogenic material embedded on an outer surface of the polymer.

4. The echogenic suture of claim 1, wherein materials of the echogenic suture comprise an inner polymer core surrounded by an echogenic material layer that is surrounded by an outer polymer layer.

5. The echogenic suture of claim 1, wherein materials of the echogenic suture comprise an inner polymer core surrounded by an echogenic material layer.

6. The echogenic suture of claim 1, wherein materials of the echogenic suture comprise an echogenic material core surrounded by a polymer layer.

7. The echogenic suture of claim 1, wherein materials of the echogenic suture comprise a strand with echogenic material braided together with strands of polymers to form a braided echogenic cord.

8. The echogenic suture of claim 1, wherein materials of the echogenic suture comprise a plurality of strands of a polymer weaved together to form a braid, wherein echogenic material is trapped in weaves of the braid.

9. The echogenic suture of claim 1, wherein materials of the echogenic suture comprise an inner core with a first density and an outer jacket with a second density, wherein a change in density between the first density and the second density provides the acute changes in material density of materials that augment the echogenic signal of the echogenic suture.

10. The echogenic suture of claim 1, wherein materials of the echogenic suture comprise one or more strands of a polymer and one or more strands of an echogenic material, the one or more strands of the polymer and the one or more strands of the echogenic material being plied together to form the echogenic suture.

11. The echogenic suture of claim 1, wherein materials of the echogenic suture comprise an ePTFE core surrounded by a jacket that includes echogenic material, the jacket formed from ribbons of a polymer with the echogenic material that are wrapped around the ePTFE core.

12. A method for performing medical procedures, the method comprising:
attaching an echogenic cord to targeted tissue of a heart, the echogenic cord including a distal anchor implant and a suture extending proximally from the distal anchor implant, the echogenic cord having an echogenic material that increases visibility to an ultrasound imaging device; and
anchoring a proximal end of the echogenic cord to the heart.

13. The method of claim 12, wherein the echogenic cord comprises an inner polymer core surrounded by an echogenic material layer that is surrounded by an outer polymer layer.

14. The method of claim 12, wherein the echogenic cord comprises a strand with echogenic material braided together with strands of polymers to form a braided echogenic cord.

15. The method of claim 12, wherein the echogenic cord comprises an inner core with a first density and an outer jacket with a second density, wherein a change in density between the first density and the second density provides an acute density change to increase ultrasound reflections off of the echogenic cord.

16. An echogenic suture for use as artificial cords in cardiac valve repairs, the echogenic suture comprising echogenic material that increases visibility to an ultrasound imaging device.

17. The echogenic suture of claim 16, wherein the echogenic suture comprises a polymer with echogenic material embedded therein.

18. The echogenic suture of claim 16, wherein the echogenic suture comprises a polymer with echogenic material embedded on an outer surface of the polymer.

19. The echogenic suture of claim 16, wherein the echogenic suture comprises a strand with echogenic material braided together with strands of polymers to form a braided echogenic suture.

20. The echogenic suture of claim 16, wherein the echogenic suture comprises a plurality of strands of a polymer weaved together to form a braid, wherein echogenic material is trapped in weaves of the braid.
